(19) United States
(12) Patent Application Publication
(10) Pub. No.: US 2011/0009623 A1
(43) Pub. Date: Jan. 13, 2011

(54) SELECTIVE GROWTH OF STABLE POLYMORPHS

(75) Inventors: Jason R. Cox, Southboro, MA (US); Marta Dabros, Worcester, MA (US); Venkat R. Thalladi, Worcester, MA (US)

Correspondence Address: BURNS & LEVINSON, LLP, 125 SUMMER STREET, BOSTON, MA, 02110, US

(73) Assignee: Worcester Polytechnic Institute, Worcester, MA (US)

(21) Appl. No.: 12/293,692

(22) PCT Filed: Mar. 20, 2007
(86) PCT No.: PCT/US07/64372

§ 371 Date: Sep. 13, 2010

Related U.S. Application Data
(60) Provisional application No. 60/783,952, filed on Mar. 20, 2006.

(51) Int. Cl. C07D 223/04; B01D 9/00; C07D 473/04; C07D 209/26; C07D 207/00; C07C 57/03; C07C 65/10; C07C 57/30; C07C 45/00

(52) U.S. Cl. 540/608; 422/245.1; 544/267; 548/501; 548/550; 562/466; 562/477; 562/496; 568/309

(57) ABSTRACT

Methods and an apparatus for producing stable crystal polymorphs are provided. Inner surfaces of glass vials with a concave bottom topography are coated with nonstick compounds to select for nucleation of stable polymorphs.

### BACKGROUND OF THE INVENTION

Many pharmaceutical solids can exist in different physical forms. Polymorphism is often characterized as the ability of a drug substance to exist as two or more crystalline phases that have different arrangements and/or conformations of the molecules in the crystal lattice. Amorphous solids consist of disordered arrangements of molecules and do not possess a distinguishable crystal lattice. Solvates are crystalline solid adducts containing either stoichiometric or nonstoichiometric amounts of a solvent incorporated within the crystal structure. If the incorporated solvent is water, the solvates are also commonly known as hydrates. Polymorphism refers to the occurrence of different crystalline forms of the same pharmaceutical substance.

Polymorphs and/or solvates of a pharmaceutical solid can have different chemical and physical properties such as melting point, chemical reactivity, apparent solubility, dissolution rate, optical and electrical properties, vapor pressure, and density. These properties can have a direct impact on the processability of drug substances and the quality/performance of drug products, such as stability, dissolution, and bioavailability. A metastable pharmaceutical solid form can change crystalline structure or solvate/desolvate in response to changes in environmental conditions, processing, or over time.

The solid state characteristics of drugs are known to potentially exert a significant influence on the solubility parameter. Polymorphs of a drug substance can have different apparent aqueous solubility and dissolution rate; when such differences are sufficiently large, bioavailability is altered and it is often difficult to formulate a bioequivalent drug product using a different polymorph.

Polymorphs of a pharmaceutical solid may have different physical and solid state chemical (reactivity) properties. The most stable polymorphic form of a drug substance is often used because it has the lowest potential for conversion from one polymorphic form to another while the metastable form may be used to enhance the bioavailability. Gibbs free energy, thermodynamic activity, and solubility provide the definitive measures of relative polymorphic stability under defined conditions of temperature and pressure. The relative polymorphic stability may be determined by an iterative examination of the relative apparent solubility of supersaturated solutions of polymorphic pairs. Since the rate of conversion to the more stable form is often rapid when mediated by the solution phase, the less stable polymorph with the greater apparent solubility dissolves, while the more stable polymorph with the lower apparent solubility crystallizes out upon standing.

Solid-state reactions include solid-state phase transformations, dehydration/desolvation processes, and chemical reactions. One polymorph may convert to another during manufacturing and storage, particularly when a metastable form is used. Since an amorphous form is thermodynamically less stable than any crystalline form, inadvertent crystallization from an amorphous drug substance may occur. As a consequence of the higher mobility and ability to interact with moisture, amorphous drug substances are also more likely to undergo solid-state reactions.

In addition, phase conversions of some drug substances are possible when exposed to a range of manufacturing processes. Milling/micronization operations may result in polymorphic form conversion of a drug substance. In the case of wet granulation processes, where the usual solvents are aqueous, one may encounter a variety of interconversions between anhydrates and hydrates, or between different hydrates.

Early discovery of thermodynamically stable drug polymorphs is critical in pharmaceutical development to avoid formulation problems and potential withdrawal of the life-saving drugs from the market. Tales of disappearing polymorphs are well-known in chemical literature; most of these tales are attributable to the late stage appearance of a thermodynamically stable polymorph that replaced the previously existing metastable form.

Understanding polymorphism gives companies a decided advantage in bringing new drugs to the marketplace. First and foremost, predicting any possible polymorphs for a drug product can be used to diminish the possibility of contamination during a drug's manufacture or storage by other polymorphic forms. Failure to catch contamination can have life-threatening consequences in some cases. Crystallizing an unintended polymorph during manufacture can mean weeks or even months of production downtime while scientists find and correct the cause of the new crystal form or go through another round of testing to obtain approval for the new form.

Second, understanding which crystal structures are possible allows researchers to maximize, in some cases, a compound's desired properties. Changes in solubility, formulation properties, processing properties, and shelf life can be achieved by switching between drug polymorphs. Understanding these factors early in the development of a new drug may mean a more active, more stable, or more cheaply manufactured drug.

### SUMMARY OF THE INVENTION

The invention provides a method of producing a stable crystal polymorph product of a substance where a substance is prepared as a supersaturated solution. The supersaturated solution is maintained in contact with a material coated with a substrate suitable for allowing nucleation of at least one crystal of a stable polymorph and then allowing the crystals to grow to a suitable size.

In one embodiment, the substrate is a nonstick coating. Examples of nonstick coatings include Teflon, silanes, thiols, superhydrophobic compounds, fluorinated polymers, polyfluoro compounds, polyfluoro polymers, perfluoro compounds, perfluoro polymers, micro-structured surfaces and nanostructured surfaces. Superhydrophobic surfaces are surfaces that have a water contact angle of 180 degrees. The surfaces used herein have a water contact angle of about 105 degrees. Thus, surfaces with non-stick coatings useful for this invention have water contact angles that range from about 100 degrees to 180 degrees.

In a one embodiment, the substrate is a silane according to the formula: R—SiCl₃, wherein R can be (CH₂)₂CO₂CH₃, (CH₂)₃CN, (CH₂)₃Cl, C₆H₄CH₂Cl, (CH₂)₉CH═CH₂, (CH₂)₁₇CH₃ or (CH₂)₂(CF)₄CF₃. In a particular aspect of this embodiment R is (CH₂)₂(CF)₄CF₃.

The substrates are coated onto materials, including but not limited to, glass, gold, silica, quartz, metal oxide, polymer, and metal. In one embodiment of the invention, a substrate is coated onto glass. In a particular aspect of this embodiment a glass vial is used, especially a glass vial with a concave bottom surface. Preferably, the vials used have a high surface-to-volume ratio, e.g. a surface-to-volume ratio between 2.37 cm⁻¹ and 5.61 cm⁻¹.

The method of the invention can be used to crystallize various substances, including but not limited to, pharmaceuticals, inorganic salts, organic compounds with hetero-atom containing functional groups, carboxylic acids, primary amides, secondary amides, tertiary amides, esters, anhydrides, halogenated compounds, nitriles, nitro-containing compounds, nitroso-containing compounds, primary alcohols, secondary alcohols, tertiary alcohols, phenols, aromatics, heterocyclic compounds, enols, ketones, aldehydes, oximes, sulfonic acids, phosphonic acids, carbohydrates, amino acids, proteins and salts of organic compounds.

In some embodiments pharmaceutical compounds are crystallized. Examples of polymorphic pharmaceutical compounds that can be crystallized by the method of the invention, include but are not limited to, theophylline, indomethacin, carbamazepine, naproxen, ibuprofen, aspirin, caffeine, nabumetone, piracetam and compounds with hetero-atom containing functional groups.

The substances being crystallized are prepared in an appropriate solution, including but not limited to, polar solvents, non-polar solvents, acetonitrile, ethanol, methanol, other alcohols, ethyl acetate, water, dimethyl formamide, dimethyl sulfoxide, ether, acetone, hexanes, benzene, toluene and xylenes.

The invention further provides an apparatus for producing stable crystal polymorph products using a glass vial wherein the interior surface of the vial is coated with a substrate. In one embodiment, the vial has a concave bottom surface.

In another embodiment, the apparatus is coated with a substrate, which is a nonstick coating, including but not limited to, Teflon, a silane, a thiol, a superhydrophobic compound, fluorinated polymers, polyfluoro compounds, polyfluoro polymers, perfluoro compounds, perfluoro polymers, micro-structured surfaces and nanostructured surfaces. Superhydrophobic surfaces are surfaces that have a water contact angle of 180 degrees. The surfaces used herein have a water contact angle of about 105 degrees. Thus, surfaces with non-stick coatings useful for this invention have water contact angles that range from about 100 degrees to 180 degrees.

In a one embodiment, the substrate is a silane according to the formula:

R—SiCl₃, wherein R can be (CH₂)₂CO₂CH₃, (CH₂)₃CN, (CH₂)₃Cl, C₆H₄CH₂Cl, (CH₂)₉CH═CH₂, (CH₂)₁₇CH₃ or (CH₂)₂(CF)₄CF₃. In a particular aspect of this embodiment R is (CH₂)₂(CF)₄CF₃.

### BRIEF DESCRIPTION OF THE DRAWINGS

FIG. 1A depicts the molecular structure of indomethacin;

FIG. 1B is a diagram of crystal growth method-1;

FIG. 1C is a diagram of crystal growth method-2;

FIG. 2 is a schematic illustration of silane substrates;

FIG. 3A is a photograph of crystal growth on a 5 monolayer-coated slide with focus on slide (method-1);

FIG. 3B is a photograph of crystal growth on a vial containing a 5 monolayer-coated slide with focus on vial bottom not covered by slide (method-1);

FIG. 3C is a photograph of crystal growth on a 9 monolayer-coated slide with focus on slide (method-1);

FIG. 3D is a photograph of crystal growth on a vial containing a 9 monolayer-coated slide with focus on vial bottom not covered by slide (method-1);

FIG. 4 is a graph of the relative amount of γ-polymorph grown on substrates 1-9 using method-1;

FIG. 5A is a photograph perpendicular to the bottom of the vial showing crystal growth on 5 monolayer;

FIG. 5B is a photograph perpendicular to the bottom of the vial showing crystal growth on 9 monolayer;

FIG. 5C is a photograph parallel to the bottom of the vial showing crystal growth on 5 (left vial) and 9 (right vial) monolayers in ethanol solution;

FIG. 5D is a photograph parallel to the bottom of the vial showing crystal growth on 5 (left vial) and 9 (right vial) monolayers in acetonitrile solution;

FIG. 6 is a schematic illustration of plasma oxidation of glass substrates and silanization with trichlorosilane derivatives;

FIG. 7A is a DSC plot of α-polymorph melting endotherm;

FIG. 7B is a DSC plot of γ-polymorph melting endotherm;

FIG. 8A is an ATR-FT IR spectrum of α-polymorph;

FIG. 8B is an ATR-FT IR spectrum of γ-polymorph;

FIG. 9A is the calculated powder X-ray diffraction pattern of α-polymorph;

FIG. 9B is the experimental powder X-ray diffraction pattern of α-polymorph fibrous material grown in a 5 vial;

FIG. 9C is the calculated powder X-ray diffraction pattern of γ-polymorph;

FIG. 9D is the experimental powder X-ray diffraction pattern of γ-polymorph crystals grown in a 9 vial;

FIG. 10 is a photograph of glass vials used for indomethacin crystal growth (a: ½ dram; b: 3 dram; c: 20 mL);

FIG. 11A is an illustration of the calculated morphology of α-polymorph of indomethacin;

FIG. 11B is a photograph of the experimental morphology of α-polymorph of indomethacin;

FIG. 11C is an illustration of the calculated morphology of γ-polymorph of indomethacin;

FIG. 11D is a photograph of the experimental morphology of γ-polymorph of indomethacin;

FIG. 12A depicts the molecular structure of carbamazepine;

FIG. 12B is an illustration of the calculated morphology of P-monoclinic carbamazepine;

FIG. 12C is an illustration of the calculated morphology of triclinic carbamazepine;

FIG. 12D is an illustration of the calculated morphology of C-monoclinic carbamazepine;

FIG. 12E is an illustration of the calculated morphology of trigonal carbamazepine;

FIG. 13A is a photograph of a negative replica of a vessel with concave topography;

FIG. 13B is a photograph perpendicular to the bottom of the vial (concave) showing crystal growth; P-monoclinic (blocks) in center and trigonal (needles) at edges;

FIG. 13C is a photograph of a negative replica of a vessel with corrugated topography;

FIG. 13D is a photograph perpendicular to the bottom of the vial (corrugated) showing crystal growth; P-monoclinic (blocks) on peaks and trigonal (needles) in troughs; and

FIG. 14 is a photograph showing the selective growth of P-monoclinic polymorph of carbamazepine in a test tube coated with 9 monolayers with uniform concave topography.

### DETAILED DESCRIPTION OF THE INVENTION

The method of the invention, crystal growth in vials exposing nonstick surfaces, is useful for widespread applications in pharmaceutical crystallization and polymorphism. Unlike the surface enabled crystal growth methodologies developed before, the method disclosed herein, does not require the knowledge of specific interfacial interactions. Thus, this new method can be applied to any solid drug even if it's structural, morphological and other physical properties are unknown. The use of this method increases the probability of finding the thermodynamically stable drug polymorphs at the early stages of pharmaceutical development. This method is also useful for the high throughput screening of crystallizations, which is widely used in the current pharmaceutical industry. In addition, the silane monolayers are robust (when compared to thiol monolayers on metal surfaces) and they are less likely to contaminate crystalline materials grown using the previously known methods. The new method, described herein, is being applied to crystallize several polymorphic drugs to test its generality and wider applicability. This method has been tested and shown to be applicable to indomethacin, an NSAID, and carbamazepine, a drug used in the treatment of epilepsy, trigeminal neuralgia and other diseases. Moreover, the method is applicable to a wide variety of pharmaceuticals that exhibit polar and hydrogen bonding functionalities. This method is also applicable to inorganic salts (e.g. KNO₃) and organic compounds (e.g. m-nitrophenol) with hetero-atom containing functional groups.

The first distinction between polymorphs occurs during nucleation. Molecular clusters must assume a critical size before they can proceed to grow into crystals. The forces on the surface of the cluster tend to dissolve the cluster, while the forces within the cluster tend to hold the cluster together. Providing an external surface with suitable steric and electronic chemistry promotes the growth of a specific polymorph. The current invention employs synthetic surfaces as substrates for phase selective crystal growth of pharmaceutical drugs.

Several researchers have used structured (thiol self-assembled monolayers, Langmuir monolayers and single crystal faces) and non-structured (silane monolayers and polymer particles) surfaces to study the crystal growth of organic and inorganic compounds. Structured surfaces exhibit long range (>100 μm²) order and periodicity, whereas non-structured surfaces do not exhibit such two-dimensional crystallinity. Polymorph selectivity is observed on structured and non-structured surfaces; chemical complementarity assisted by some level of geometric complementarity at the growth interface is assumed to be responsible for the observed selectivity. For an overview of polymorphism in pharmaceutical compounds, see: Polymorphism in Pharamaceutical Solids (Ed.: H. G. Brittain), Marcel Dekker, New York (1999); S. R. Byrn et al., Solid-State Chemistry of Drugs, 2nd ed., SSCI, West Lafayette (1999); J. Bernstein, Polymorphism in Molecular Crystals, Oxford University Press, New York (2002); Polymorphism in the Pharmaceutical Industry (Ed.: R. Hilfiker), Wiley-VCH, Weinheim (2006); the entire contents of which are incorporated herein by reference.

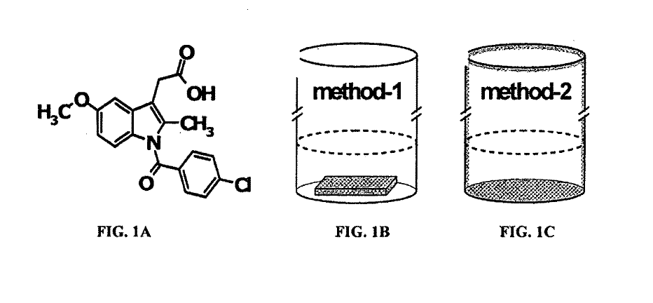

Traditionally, monolayers formed on glass or gold surfaces are immersed in a saturated solution of the pharmaceutical. This method of crystal growth is referred to herein as method-1. FIG. 1b is a diagram of crystal growth method-1; the gray shading indicates monolayers and dashed lines indicate the air-solution interface.

In the instant invention, the improved method of crystal growth ensures surface induced nucleation. Monolayers were formed directly on the inside surface of a vial, preferably a glass vial, and the vial was filled with a saturated pharmaceutical solution. Using this method, minimized edge effects and the only available sites for nucleation are on the desired surface or in solution. Bulk nucleation is minimized by using vials with relatively small diameters. This method of crystal growth is referred to herein as method-2. FIG. 1e is a diagram of crystal growth method-2; the gray shading indicates monolayers and dashed lines indicate the air-solution interface.

Polymorph growth is influenced by heterogeneous nucleation. From an understanding of the structures of growing crystal face and substrate (SAM), it is possible to “design” new surfaces that can generate desired polymorphs. In one embodiment of the invention, perfluoroalkyl terminated silane monolayers promoted the exclusive growth of the stable polymorph (γ-form) of indomethacin. This selective growth is promoted not by the enhanced nucleation of the γ-form, but by the suppressed nucleation of the metastable polymorph (α-form). The silane monolayers fabricated on the surfaces of glass vials (as opposed to monolayers fabricated on glass slides) minimized concomitant crystallization of polymorphs.

In the present invention, thiol and silane self-assembled monolayers (SAMs) formed on gold or glass bases were used as synthetic substrates to explore the polymorphism of theophylline (a bronchodilator), indomethacin (an NSAID), and carbamazepine (an anticonvulsant).

Theophylline: Theophylline exists as an anhydrous and a monohydrate polymorph. Hydrophilic thiol SAMs exposing carboxy and hydroxy groups promoted the selective growth of the anhydrous form of theophylline, whereas hydrophobic SAMs allowed the growth of the monohydrate. Experimental and computational analysis showed that (200) faces of the anhydrous form have the best coincident epitaxy and highest chemical complementarity with hydrophilic SAMs.

Indomethacin: Indomethacin is an anti-pyretic anti-inflammatory drug (NSAID). It has five true polymorphs but only two, forms I (γ) and II (α), are regularly obtainable. The others are metastable and readily transform to the γ-form or α-form on standing or heating. The γ-form is thermodynamically most stable and displays a well-defined morphology of rhombic plates. Crystals of the α-form grow as undefined fibrous structures with needle-like morphology.

The α-form (less stable form) and γ-form (stable form) of indomethacin, crystallize concomitantly in the absence of a SAM. Silane SAMs exposing chloro functionalities promoted the crystal growth of the α-form, whereas perfluoro SAMs allowed the growth of the γ-form.

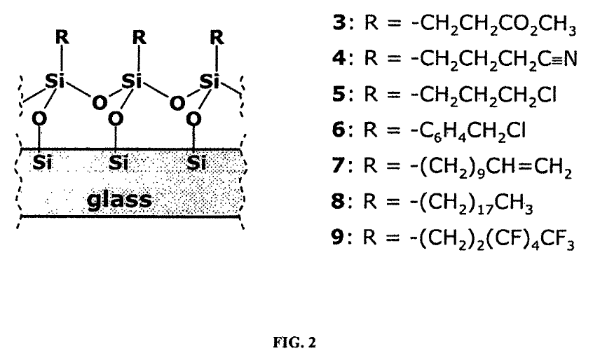

Indomethacin possesses several functionalities (carboxy, tertiary amido, methoxy, chloro: see FIG. 1a). Silane monolayers bearing different functional groups (3-9, FIG. 2) were used to examine the effects on indomethacin crystal growth. In all the experiments, bare glass (1) and plasma oxidized glass (2) substrates were used as controls.

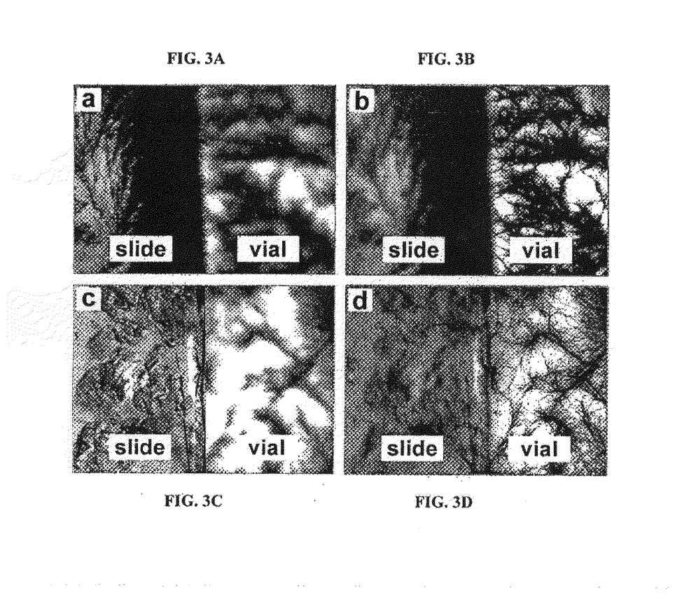

Indomethacin crystallizes concomitantly as α and γ polymorphs from ethanol. The two polymorphs have distinct crystal structures (α: P2₁, Z′=3, γ: P 1̅, Z′=1), melting points (α: 153° C., γ: 158° C.) and morphologies (α: needles, γ: plates). They can be identified by powder X-ray diffraction (PXRD), IR spectroscopy, differential scanning calorimetry (DSC) and optical microscopy. First, studies were done on the effect of silane substrates 1-9 on the crystal growth of indomethacin using growth method-1 (FIG. 1b). In this method, silane monolayers were prepared on glass slides, these slides were placed into glass vials containing ethanolic solutions of indomethacin, and crystals were grown by slow evaporation of the solvent. Repeated experiments using method-1 and substrates 1-9 led to three consistent results: (i) both α and γ-polymorphs crystallize concomitantly on substrates 1-8 (FIG. 3a); (ii) on substrate 9, crystals of γ-polymorph are formed predominantly (FIG. 3c); and (iii) crystals of α-polymorph are always formed on the vial surfaces (FIGS. 3b and 3d), especially on the walls of the vials.

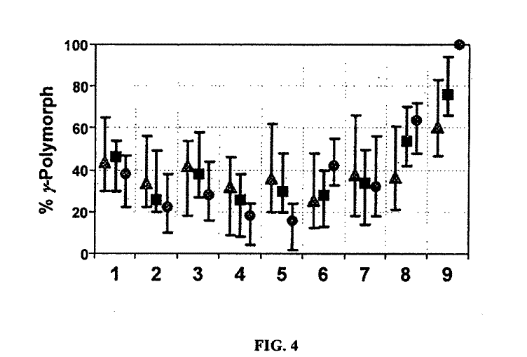

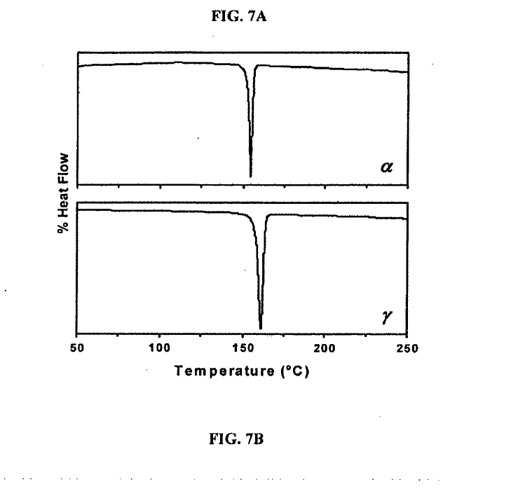

FIG. 4 shows the relative amount of the γ-polymorph obtained on substrates 1-9 in eight different experiments. Initially, α and γ-polymorphs were characterized by PXRD, IR spectroscopy and DSC (FIGS. 7A-9D). Later, optical microscopy was used to distinguish between the two forms. The crystals were separated from the slides and vials, and weighed on an analytical balance. Though this method is approximate, it is rapid and avoids the co-grinding of samples, which may result in the unintended phase transition between the two polymorphs. Such co-grinding is required for the quantification by DSC, PXRD and IR spectroscopy.

The analysis of the total solid material in each vial (FIG. 4, triangles) shows that the amount of γ-polymorph in vials containing 9 slides is greater compared to other vials. This result becomes more prominent when the analysis is restricted only to the material that is present on the slides (FIG. 4, squares). These findings suggest that 9 surfaces may be responsible for the enhanced growth of the γ-polymorph. Nucleation of the α-polymorph on vial surfaces (and probably also in bulk solution) is responsible for the concomitant crystallization of the two polymorphs in vials containing 9 slides.

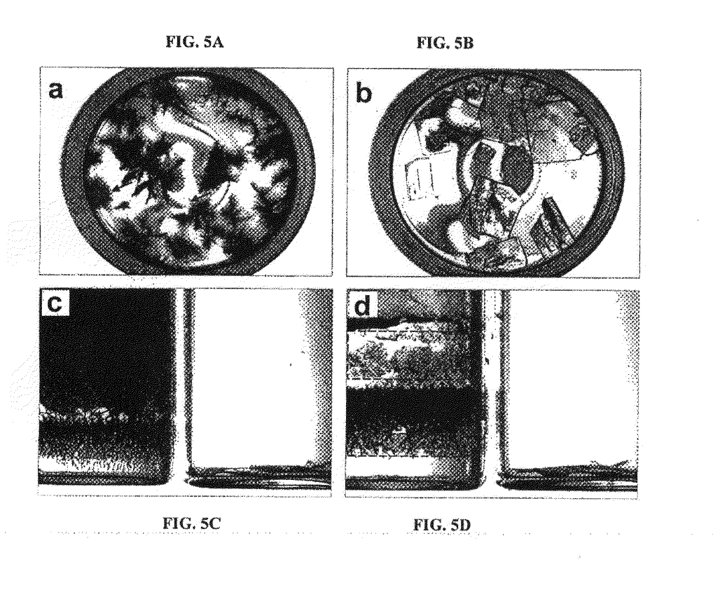

In order to eliminate the competing influence of two different surfaces on the crystal growth, the silane monolayers were fabricated directly on the inner surfaces of the glass vials and these vials were then used for crystal growth (FIG. 5). This is growth method-2 (FIG. 1c); circles in FIG. 4 show the results from this method. Arrows and boxed areas in FIGS. 5a-d show some of the crystals of γ-polymorph crystallized along with the γ-polymorph. While a mixture of two polymorphs crystallizes in control vials 1-2 and vials functionalized with monolayers 3-8 (with some differences in the relative amounts of the two forms), vials coated with 9 monolayers yielded the γ-polymorph exclusively in eight different experiments. Similar results were obtained when crystallizations were carried out in acetonitrile or other altered conditions. The crystal growth experiments described so far used ethanol solutions made from γ-polymorph of indomethacin. Crystallizations from ethanol solutions that were made from α-polymorph were also performed. In separate experiments, crystallizations were performed by evaporating ethanol at a much slower rate and also at 0° C. In all these cases, similar results were obtained: (a) crystals of α-polymorph always grew on the walls of vials 1-8; (b) no crystal growth occurred on the walls of 9 vials; and (c) crystals of only γ-polymorph were grown in 9 vials.

The following experimental observations explain the preferential and exclusive growth of the γ-form in vials containing perfluorinated monolayers. (1) Except in vials functionalized with 9 monolayers, crystals of mostly α-polymorph grew on the vial walls as the solvent-front evaporated (FIG. 5 c-d). (2) Crystals (of γ-polymorph) appeared after a significantly longer time interval in 9 vials. When crystallizations were carried out with 25 mM ethanolic solutions of indomethacin at 20° C., crystals of α-polymorph appeared on the walls of vials 1-8 in 14-20 hours whereas the crystals of γ-polymorph appeared at the bottom of the 9 vials in 30-56 hours (FIG. 5b). (3) The total number of crystals in a given 9 vial is much smaller than in other vials. In addition, individual crystals are larger and well-formed in 9 vials.

These observations indicate that the crystal growth of α- and γ-polymorphs follows the Ostwald's rule of stages. Accordingly, the nuclei of α-form (kinetic polymorph) are formed in preference to the γ-form (thermodynamic polymorph) at the onset of saturation. Crystals of α-polymorph grow on non-perfluorinated substrates 1-8 because these substrates allow the adsorption of α-nuclei on their surfaces and promote the heterogeneous nucleation of α-form. In contrast, the perfluoroalkyl monolayers 9 inhibit the attachment of kinetic nuclei to the surface, thereby suppressing their heterogeneous nucleation and further crystal growth of the α-polymorph. With time, however, the nuclei of thermodynamic γ-polymorph are formed in solution; as the solvent evaporates crystals of γ-polymorph grow while the nuclei of α-polymorph dissolve in solution. This phenomenon, the growth of a stable form at the expense of a metastable form, is known as Ostwald ripening. The nonstick nature of perfluoroalkyl surfaces (e.g., Teflon) is well established. Here, nonstick surfaces were used to thwart the nucleation of the metastable polymorph; in so doing, these surfaces promote the growth of the stable polymorph.

Carbamazepine: Carbamazepine, an anticonvulsant pharmaceutical drug used in epilepsy, trigeminal neuralgia and other diseases, exists as four anhydrous polymorphs and a hydrate. Recently several solvates and cocrystals of this drug have been prepared.

The four anhydrous polymorphs of carbamazepine have been variously named. Herein the nomenclature used is that given by A. L. Grzesiak, M. Lang, K. Kim, and A. J. Matzger in Journal of Pharmaceutical Sciences (2003, 92, 2260). According to this nomenclature, the four polymorphs are called P-monoclinic (space group P2₁/c or P2₁/n), triclinic (space group P _{1̅}), C-monoclinic (space group C2/c), and trigonal (R _{3̅}). The stability of these polymorphs is in this order: P-monoclinic>triclinic>C-monoclinic>trigonal. Traditionally, the P-monoclinic and trigonal polymorphs can be grown from ethanol solutions at 20-25° C. and below 10° C. respectively. The triclinic form is usually obtained from the melt, and the C-monoclinic form is grown in the presence of some specific polymeric additives.

The nonstick surfaces (formed by the 9-monolayers) promoted the growth of the stable P-monoclinic polymorph at conditions (8-10° C.) conducive for the growth of trigonal polymorph. A range of other surfaces (formed by 3-8 monolayers) yielded either the trigonal polymorph or the concomitant growth of the two polymorphs. The crystal growth conditions for carbamazepine are the same as those used in indomethacin, except for the differences in growth temperature (8-10° C.).

One unique feature in the crystal growth of carbamazepine that is not seen in indomethacin is that vials with uneven and corrugated bottoms led to the concomitant growth of the two polymorphs (P-monoclinic and trigonal), whereas the test tubes with uniform concave topography led to the exclusive growth of one or the other polymorph depending on the nature of the growth surface. When 9 monolayers are used, crystals of only the P-monoclinic polymorph are formed (at 8-10° C.). When plasma-treated test tubes (exposing silanol functional groups) are used, the trigonal polymorph is obtained (at 8-10° C.). These results indicate a new invention that the nonstick surfaces formed on vessels with uniform topography are better suited for the crystal growth of the stable polymorph in the presence of the 9 monolayers or other similar nonstick surfaces.

The effect of topography on the site-selective crystal growth of different polymorphs can be explained on the basis of Ostwald ripening and the nonstick nature of the 9 monolayers. At the outset of crystallization, that is at the beginning of supersaturation, nuclei of kinetic (trigonal) polymorph are expected to be formed in large numbers. Further growth of these nuclei into crystals is severely hampered by the disfavored heterogeneous nucleation at the nonstick surfaces created by 9 monolayers. As the evaporation progresses, the prolonged time enables the growth of the nuclei of the thermodynamic polymorph (P-monoclinic). The nuclei of the stable polymorph, once formed, continue to grow at the expense of (that is, at the depletion of) the nuclei of kinetic polymorph. Slowly, the nuclei of the stable polymorph continue growing into larger and larger crystals while the nuclei of kinetic polymorph continue to deplete and stay in (now) highly supersaturated solutions. If the bottom of the vessels is uneven (or corrugated) this highly supersaturated solution is now confined to the crevices of the uneven bottom surface. At some point this solution loses the contact with the crystal of the stable polymorph (now sitting on a hill on the uneven surface); that is, at this stage, the transformation of the kinetic polymorph into the thermodynamic polymorph is no longer viable. At this stage, the crystals of the less stable kinetic polymorph are grown within the crevices (around the stable crystals) of bottom of the vessel from a highly supersaturated solution. Such loss of contact (of the crystallization solution) with the stable crystals is prohibited by choosing vessels with uniform concave bottoms; hence only the crystals of stable polymorph is obtained in vessels with uniform concave bottoms.

Thus, engineered vessel topographies combined with surface modifications can be used as an effective tool to grow the crystals of stable polymorph in preference to the less stable polymorphs (under conditions where the less stable polymorphs normally grow).

### EXAMPLES

Materials. (2-Carbomethoxy)ethyltrichlorosilane (3) was purchased from Oakwood Products Inc. and used as received. (3-Cyanopropyl)trichlorosilane (4) and (1H,1H,2H,2H-perfluorooctyl)trichlorosilane (9) were purchased from Aldrich and used without further purification. (3-Chloropropyl)trichlorosilane (5), (4-chloromethyl)phenyltrichlorosilane (6), and indomethacin were purchased from Alfa Aesar and used without further purification. 10-Undecenyltrichlorosilane (7) was purchased from Gelest Inc. and used as received. n-Octadecyltrichlorosilane (8) was purchased from TCI America and used as received. Absolute ethanol and HPLC grade toluene were purchased from Pharmco and used as received. Precleaned 25×75×1 mm and 50×75×1 mm glass microscope slides were purchased from VWR and ½ dram (1.85 mL), 3 dram (11.09 mL) and 20 mL precleaned glass vials were purchased from Kimble and Wheaton Scientific and used as received. The vials were sold in these different denominations (dram and mL); in the following sections we refer to the vials using the naming given above.

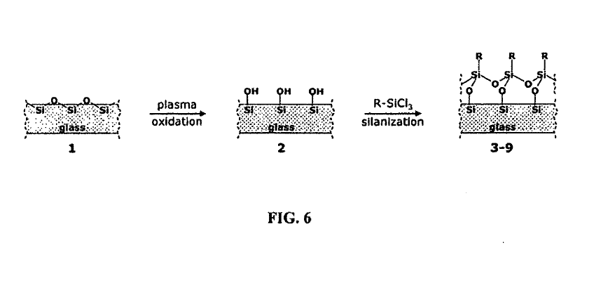

Preparation of Substrates and Plasma Oxidation. Glass Microscope Slide Substrates were prepared by cutting the slides into 1×10×15 mm strips. These strips and glass vials (to be used as silane substrates) were oxidized for approximately two minutes under an oxygen plasma using a plasma etcher (SPI Plasma Prep II) that was operating at 13.56 MHz under a 200 micron vacuum. Plasma oxidation of glass substrates is a well established process; it creates surfaces exposing silanol groups (FIG. 6). After the completion of plasma oxidation, the mild vacuum inside the plasma chamber was maintained (to avoid contamination from outside moisture) until the glass slides and vials were ready for monolayer deposition. All the substrates (slides and vials) were oxidized immediately prior to monolayer deposition.

Fabrication of Silane Monolayers on Class Slide Substrates. Trichlorosilane (R—SiCl₃) solutions (˜1 mM) were freshly prepared in toluene and transferred to 20 mL glass vials. Freshly oxidized glass slide strips were removed from the plasma etcher and immersed in the trichlorosilane solutions. The glass vials were completely filled with the silane solutions; they were capped and stored in a cabinet for approximately three hours. The slides were removed from the trichlorosilane solutions, rinsed thoroughly with toluene, and sonicated for 20 minutes in acetone using a Branson 2510 sonicator. After the sonication, the slides were washed with absolute ethanol at least three times and dried under a stream of nitrogen. These slides exposing the silane monolayers at the surface (FIG. 6) were used for crystal growth within 30 minutes of the fabrication of the monolayers.

Fabrication of Silane Monolayers on the Inner Surfaces of Glass Vials. Freshly prepared ˜1 mM toluene solutions of trichlorosilanes were transferred to oxidized ½ dram glass vials that had just been removed from the plasma chamber. The vials were filled completely with silane solutions, capped, and stored in a cabinet. After three hours, the trichlorosilane solutions were pipetted out of the vials using glass Pasteur pipettes. The vials were rinsed thoroughly with toluene, sonicated for 20 minutes in acetone using a Branson 2510 sonicator, washed at least three times with absolute ethanol and dried under a stream of nitrogen. These vials now contained silane monolayers on their inner surfaces (FIG. 6); they were used for crystal growth experiments within 30 minutes of the fabrication of the monolayers.

Contact Angle Measurements. Contact angles were measured at nine different positions for each type of surface (three separate slides) with a manual goniometer (Rame-Hart, Model 100-00). The values reported in Table 1 were averages of these measurements. Deionized water droplets (3 μL) were added to each surface using a calibrated Epindorf pipette and the angles obtained had a maximum error of ±2.3°. The contact angles show that the surface is modified; they provide a rough measure of hydrophobicity and hydrophilicity of the surfaces.

TABLE 1
Contact angle data for substrates 1-9.

| Substrate | Silane | Contact Angle |
|---|---|---|
| 1 | bare glass | 19.3 ± 2.3° |
| 2 | plasma treated glass | 13.5 ± 2.1° |
| 3 | (2-carbomethoxy)ethyltrichlorosilane | 42.1 ± 1.8° |
| 4 | (3-cyanopropyl)trichlorosilane | 56.3 ± 2.2° |
| 5 | (3-chloropropyl)trichlorosilane | 68.4 ± 1.8° |
| 6 | (4-chloromethyl)phenyltrichlorosilane | 75.8 ± 1.4° |
| 7 | 10-undecenyltrichlorosilane | 86.7 ± 1.8° |
| 8 | n-octadecyltrichlorosilane | 91.3 ± 1.2° |
| 9 | (1H,1H,2H,2H-perfluorooctyl)trichlorosilane | 104.4 ± 1.1° |

Crystal Growth on Glass Slides Bearing Silane Monolayers. In a 100 mL beaker, 25 mM indomethacin solution was made in ethanol and heated at 60° C. for 30 minutes. Ethanol was added in excess at the beginning; the volume of the solution was reduced to required concentration by the evaporation of solvent during heating. The solution was cooled to 20° C. and filtered to 20 mL glass vials containing glass slides bearing silane monolayers. The slides were placed at the bottom of the vial as shown in FIG. 1b. Each vial was filled with 5 mL of the solution and covered with a perforated aluminum foil to allow the evaporation of the solvent. All the crystal growth experiments were performed at 20° C. in parallel for at least eight times. The results in all these experiments were qualitatively similar; see FIG. 4 for the quantification of the results. Crystals of α-polymorph appeared on the vial walls within 10-20 hours in all the cases.

Crystal Growth in Glass Vials Functionalized with Silane Monolayers on the Inner Surfaces. Indomethacin solutions (25 mM) were prepared as above and filtered to ½ dram glass vials functionalized with silane monolayers. Each vial was filled with 1.2 mL of the solution and covered with a perforated aluminum foil to allow the slow evaporation of the solvent. All the crystal growth experiments were performed at 20° C. in parallel for at least eight times. The results in all these experiments were qualitatively similar; see FIG. 4 and below for the quantification of the results. Crystals of α-polymorph appeared on the walls of vials 1-8 within 14-20 hours in all the cases. In 9 vials, crystals of γ-polymorph appeared at the bottom of the vials in 30-56 hours. In these vials crystal growth did not occur on the vial walls. Commercial indomethacin contained predominantly (>97%) γ-polymorph. Crystallizations were also done in 9 vials using indomethacin solutions that were prepared from >99% α-polymorph. In three out of three experiments under these conditions, we observed the exclusive crystal growth of γ-polymorph in 9 vials.

Crystal Growth in Functionalized Glass Vials from Acetonitrile Solutions. The procedure is as above except that acetonitrile is used as a solvent instead of ethanol. The concentrations of the solutions were 25 mM. As in the case of ethanol solutions, control vials 1-2 and vials functionalized with monolayers 3-8 yielded a mixture of α- and γ-polymorphs, whereas the vials functionalized with 9 monolayers produced only the γ-polymorph. These experiments were performed in duplicate.

Crystal Growth by Slower Evaporation in Functionalized Glass Vials. Experiments using 25 mM ethanol solutions in functionalized ½ dram vials were done in duplicate. The vials were sealed with parafilm and a pinhole was made in the parafilm with the tip of a needle. The objective here was to retard the rate of evaporation of ethanol and contrast these results with the experiments above. As above, crystals of α-polymorph grew on the walls of vials 1-8 and no crystal growth occurred on the walls of 9 vials. The difference in this case is that α-crystals appeared on the walls of vials 1-8 after at least four days. In 9 vials, γ-crystals appeared at the bottom after five to six days. These results confirm that the nuclei of α-polymorph survive in solution for at least four days by sticking to the walls of vials 1-8; they do not survive in 9 vials because they cannot attach to the surfaces in these vials. In other words, the growth of γ-polymorph in 9 vials is facilitated by the suppression of heterogeneous nucleation of α-polymorph. We also carried out crystallizations of indomethacin from 25 mM ethanol solutions in functionalized ½ dram vials at 0° C. In these experiments, crystals of α-polymorph grew on the walls of vials 1-8 after 10 days and crystals of γ-polymorph appeared at the bottom of the 9 vials after two weeks. Again, these results clearly point to the ability of perfluoroalkyl surfaces to thwart the heterogeneous nucleation of metastable polymorph of indomethacin.

Differential Scanning Calorimetry. These measurements were carried out with DSC-2920 (TA Instruments) in hermetically sealed and crimped aluminum pans. Samples were subjected to heating in the range 30-250° C. at a rate of 10° C. per minute (FIG. 7). The two polymorphs showed distinct endotherms corresponding to their melting temperatures; α at 153° C. and γ at 158° C. These melting points are 2-3° less than the reported values in the literature. No other phase transitions were observed in the temperature range used.

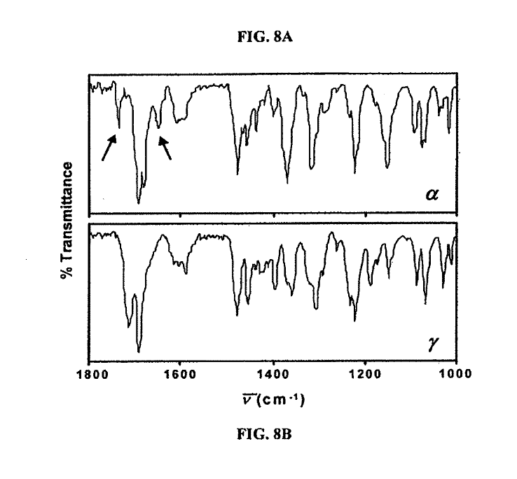

Infrared Spectra of Polymorphs. Infrared spectra were collected with a Nexus FT-IR spectrometer (Model 670) equipped with a liquid nitrogen cooled MCTA detector and an ATR accessory. IR was used as the first characterization tool because the ATR accessory allowed rapid data acquisition (<1 min) with a small amount of sample (<5 mg). The two polymorphs under consideration can be clearly identified from the IR spectra (FIG. 8). The α-polymorph crystallizes in a noncentrosymmetric space group (P2₁) with three molecules in the asymmetric unit, whereas the γ-polymorph belongs to a centrosymmetric space group (P _{1̅}) with one molecule in the asymmetric unit. Consequently the α-polymorph has greater number IR absorptions than the γ-polymorph. A comparison of the two spectra reveals that there are several peaks that distinguish the two polymorphs; the arrows in FIG. 8 indicate the characteristic absorptions used by other researchers to identify the α-polymorph.

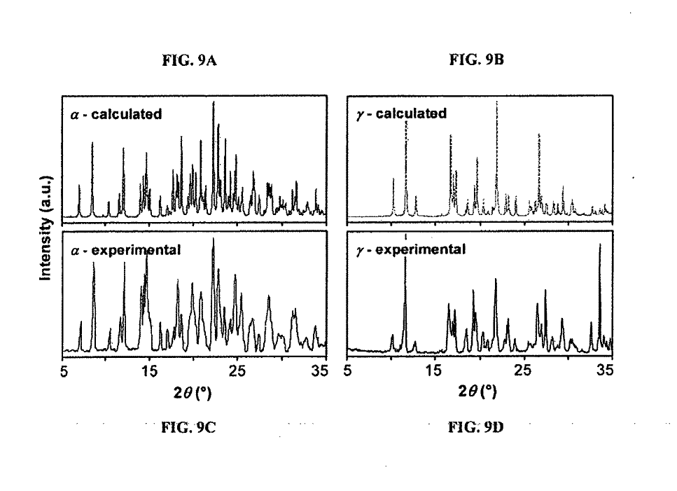

Powder X-Ray Diffraction Analysis. Powder X-ray data were collected on a Rigaku Geigerflex D-MAX/A diffractometer using Cu-Kα radiation. The instrument was equipped with a vertical goniometer and a scintillation counter as a detector and applied Bragg-Brentano geometry for data collection. X-rays were generated at a power setting of 35 kV and 35 mA. Crystals of the α-polymorph were fluffy and small quantity of this polymorph occupied large volumes; the diffraction peaks of this polymorph were usually weaker than the γ-polymorph. The crystals obtained from the experiments above were pulverized using a mortar and a pestle prior to diffraction analysis. Finely ground powders were transferred to a glass sample holder that had loading dimensions 1.6 cm×2 cm and exposed to X-rays over the 2θ range 5-40° in 0.05° steps and at a scan rate of 2° per minute. FIG. 9 shows the experimental powder patterns along with powder patterns calculated from the single crystal X-ray structures. These X-ray patterns show that the crystals obtained from 9 vials correspond to the γ-polymorph.

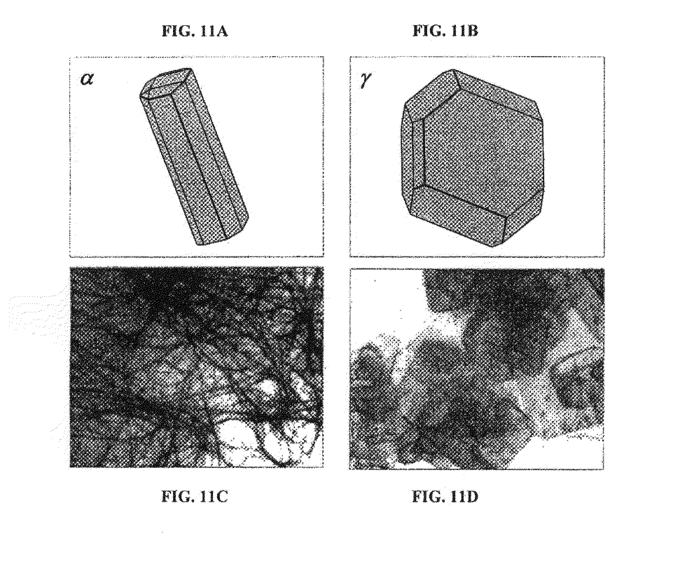

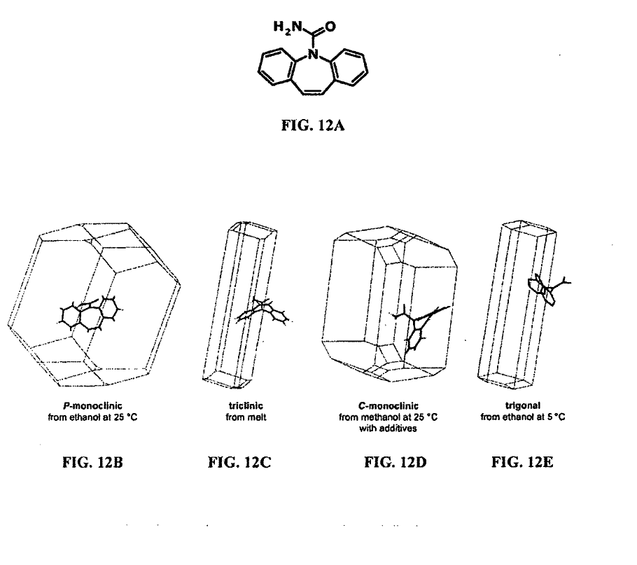

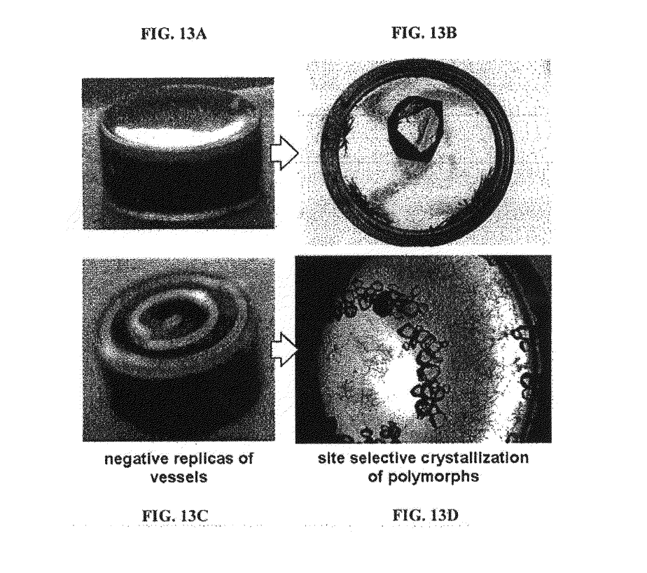

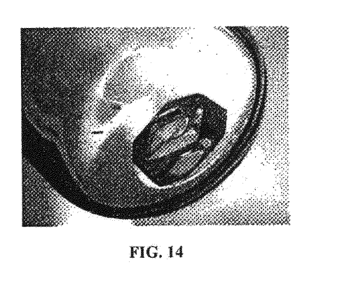

Quantitative Analysis of α- and γ-polymorphs. The crystals of α- and γ-polymorphs have distinct morphologies (see FIG. 11); the two forms are readily distinguished by visual inspection. The crystals of γ-polymorph grown on glass slides and vials were separated with the aid of a pair of tweezers, a surgical blade and microscope. The solid material from the vial was scraped onto a glass slide (50×75 mm), the crystals were spread and sorted, and the γ-crystals were moved to a different slide. These separated samples were then weighed on an analytical balance and the weights so obtained were used to calculate the relative amounts of the two polymorphs (FIG. 4). Separation of this kind invariably left a small portion of α-form in the pile of γ-form and vice versa. This method is thus approximate and cannot be used for accurate quantitative analysis. The main result of the current study (exclusive growth of the crystals of γ-polymorph on 9 monolayers), however, is unaffected by the inaccuracies of this method. Co-grinding of samples is a prerequisite for the quantification by PXRD and IR spectroscopy; such co-grinding can lead to phase transition between the polymorphs or transition to the amorphous form.

Effect of Surface-to-Volume (S/V) Ratio of the Vial on the Crystal Growth. All the experiments described of crystallizations in functionalized vials were performed using ½ dram vials. Owing to their small sizes, these vials have a high S/V ratio (5.61 cm⁻¹); crystallization in these vials is governed predominantly by heterogeneous nucleation on the surfaces (as opposed to the bulk nucleation in solution). The result in the case of 9 vials is that the nonstick perfluoroalkyl surfaces control the crystallization process: they prevent the attachment of nuclei of α-polymorph to vial walls and force the bulk nucleation of stable γ-polymorph (and further crystal growth of the stable polymorph at the expense of metastable α-polymorph by Ostwald ripening).

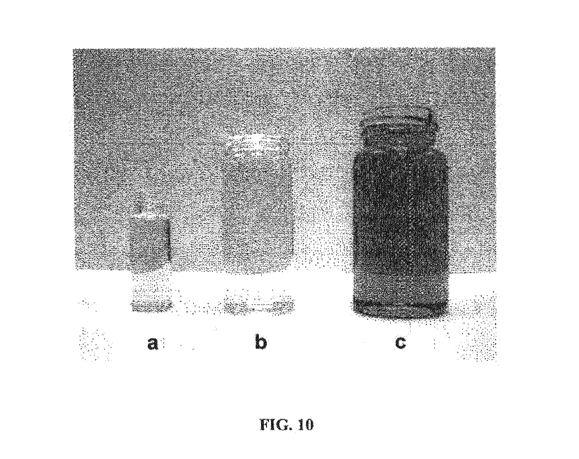

Crystal growth experiments were performed in larger vials with smaller S/V ratios (FIG. 10). Seven of the eight crystallization experiments in 3 dram vials (S/V=2.83 cm⁻¹) functionalized with 9 monolayers gave only the γ-polymorph. In one experiment, α-polymorph (6%) crystallized along with the γ-form. When 20 mL vials (S/V=2.37 cm⁻¹) functionalized with 9 monolayers were used, crystals of α-polymorph appeared (4-13%) in six of the eight experiments. Two crystal growth experiments done in 100 mL beakers (S/V=1.21 cm⁻¹) functionalized with 9 monolayers yielded α-polymorph (18% and 24%) along with the γ-polymorph in both the experiments. Thus, the current method is effective when vials with high S/V ratios are used for crystal growth. The current work shows that researchers aiming to crystallize stable polymorphs will have higher chances of success at their attempts if they use narrow tubular vessels (with high values of S/V ratios) functionalized with 9 or other related perfluoroalkyl monolayers. Similar results can be obtained when vessels with nonstick coatings or vessels made of Teflon or other related materials are used for crystal growth. Fabrication of superhydrophobic surfaces is a thriving area of current research; application of these superhydrophobic surfaces for crystal growth are also useful in the method of the current invention. Superhydrophobic surfaces are surfaces that have a water contact angle of 180 degrees. The surfaces used herein have a water contact angle of about 105 degrees. Thus, surfaces with non-stick coatings useful for this invention have water contact angles that range from about 100 degrees to 180 degrees.

Experimental and Calculated Morphologies of α- and γ-polymorphs. As stated previously, α- and γ-polymorphs have distinct morphologies. In the experiments disclosed herein, crystals of α-polymorph grew as very thin, fibrous needles and they usually appeared as clumps. In contrast, crystals of γ-polymorph grew as pseudohexagonal or nearly rectangular plates. Seldom, they also crystallized as rectangular needles or blocks. Crystals of γ-form also often grew as small clusters. This clustering of the crystals of both polymorphs and the stark differences in their morphologies allowed the easy separation of one polymorph from another by hand. FIG. 11 shows experimental morphologies of both the polymorphs along with the morphologies calculated using BFDH (Bravais-Friedel-Donnay-Harker) theory.

What is claimed is:

1. A method of producing a stable crystal polymorph product of a substance comprising the steps of:
a) preparing a solution of said substance;
b) supersaturating said solution;
c) preparing a substrate by coating a material;
d) contacting said supersaturated solution with said substrate;
e) maintaining said substance in solution for a period of time sufficient to induce the nucleation of at least one crystal of a stable polymorph; and
f) growing crystals of said stable polymorph.

2. The method of claim 1, wherein said substrate is a nonstick coating selected from the group consisting of Teflon, a silane, a thiol, a superhydrophobic compound, fluorinated polymers, polyfluoro compounds, polyfluoro polymers, perfluoro compounds, perfluoro polymers, micro-structured surfaces and nanostructured surfaces.

3. The method of claim 2, wherein said nonstick coating has a water contact angle ranging from about 100 degrees to 180 degrees.

4. The method of claim 1, wherein said substrate is a silane according to the formula: R—SiCl₃, wherein R is selected from the group consisting of (CH₂)₂CO₂CH₃, (CH₂)₃CN, (CH₂)₃Cl, C₆H₄CH₂Cl, (CH₂)₉CH═CH₂, (CH₂)₁₇CH₃ and (CH₂)₂(CF)₄CF₃.

5. The method of claim 4, wherein R is (CH₂)₂(CF)₄CF₃.

6. The method of claim 1, wherein said material is selected from the group consisting of glass, gold, silica, quartz, metal oxide, polymer, and metal.

7. The method of claim 6, wherein said material is glass.

8. The method of claim 7, wherein said glass forms the interior surface of a vial.

9. The method of claim 8, wherein said vial has a concave bottom surface.

10. The method of claim 8, wherein said vial has a high surface-to-volume ratio.

11. The method of claim 10, wherein said surface-to-volume ratio is between 2.37 cm⁻¹ and 5.61 cm⁻¹.

12. The method of claim 10, wherein said surface-to-volume ratio is at least 2.83 cm⁻¹.

13. The method of claim 10, wherein said surface-to volume ratio is 5.61 cm⁻¹ or greater.

14. The method of claim 1, wherein said substance is selected from the group consisting of pharmaceuticals, inorganic salts, organic compounds with hetero-atom containing functional groups, carboxylic acids, primary amides, secondary amides, tertiary amides, esters, anhydrides, halogenated compounds, nitriles, nitro-containing compounds, nitroso-containing compounds, primary alcohols, secondary alcohols, tertiary alcohols, phenols, aromatics, heterocyclic compounds, enols, ketones, aldehydes, oximes, sulfonic acids, phosphonic acids, carbohydrates, amino acids, proteins and salts of organic compounds.

15. The method of claim 1, wherein said solution is selected from the group consisting of polar solvents, non-polar solvents, acetonitrile, ethanol, methanol, other alcohols, ethyl acetate, water, dimethyl formamide, dimethyl sulfoxide, ether, acetone, hexanes, benzene, toluene and xylenes.

16. The method of claim 14, wherein said pharmaceutical is selected from the group consisting of theophylline, indomethacin, carbamazepine, naproxen, ibuprofen, aspirin, caffeine, nabumetone, piracetam and compounds with hetero-atom containing functional groups.

17. An apparatus for producing a stable crystal polymorph product of a substance comprising a glass vial wherein the interior surface of said vial is coated with a substrate.

18. The apparatus of claim 18, wherein said vial has a concave bottom surface.

19. The apparatus of claim 17, wherein said substrate is a nonstick coating selected from the group consisting of Teflon, a silane, a thiol, a superhydrophobic compound, fluorinated polymers, polyfluoro compounds, polyfluoro polymers, perfluoro compounds, perfluoro polymers, micro-structured surfaces and nanostructured surfaces.

20. The apparatus of claim 18, wherein said nonstick coating has a water contact angle ranging from about 100 degrees to 180 degrees.

21. The apparatus of claim 17, wherein said substrate is a silane according to the formula: R—SiCl₃, wherein R is selected from the group consisting of (CH₂)₂CO₂CH₃, (CH₂)₃CN, (CH₂)₃Cl, C₆H₄CH₂Cl, (CH₂)₉CH═CH₂, (CH₂)₁₇CH₃ and (CH₂)₂(CF)₄CF₃.

22. The apparatus of claim 20, wherein R is (CH₂)₂(CF)₄CF₃.
